(19) United States
(12) Patent Application Publication
(10) Pub. No.: US 2025/0000760 A1
(43) Pub. Date: Jan. 2, 2025

(54) EMULSION COMPOSITIONS CONTAINING PULLULAN COMPOUND AND ACRYLAMIDE FILM FORMER

(71) Applicant: L'OREAL, Paris (FR)

(72) Inventors: Brady ZARKET, Edison, NJ (US); Yixin YANG, Union, NJ (US)

(21) Appl. No.: 18/345,499

(22) Filed: Jun. 30, 2023

(51) Int. Cl. A61K 8/06; A61K 8/67; A61K 8/81; A61K 8/89; A61K 8/92; A61Q 1/02

(52) U.S. Cl. CPC A61K 8/064; A61K 8/673; A61K 8/8158; A61K 8/89; A61K 8/92; A61Q 1/02; A61K 2800/43; A61K 2800/48

(57) ABSTRACT

The invention relates to water-in-oil (w/o) emulsion compositions, preferably for skin, including at least one hydrophobically-modified pullulan compound and at least one aqueous phase film former, as well as to methods and kits including such emulsion compositions in container(s) or in application.

### FIELD OF THE INVENTION

The present invention relates to water-in-oil (w/o) emulsions (compositions) comprising at least one hydrophobically-modified pullulan compound and at least one aqueous phase film former, as well as to methods and kits comprising such emulsion compositions in container(s) or in application. The compositions have beneficial cosmetic properties including good or improved wear, water and/or sebum resistance, and/or transfer resistance properties, in particular sebum resistance.

### DISCUSSION OF THE BACKGROUND

At the present time on the market for caring for and making up keratin materials, many products claim staying power throughout the day, withstanding external factors such as water, sebum, mechanical friction, etc. (waterproof mascara, food-proof lipsticks, long-lasting foundations). Long-lasting products for the lips, the eyelashes, the eyebrows or the face, which can be used at home, are mainly based on synthetic coating polymers in the presence of organic solvents. For making up the lips and compositions for making up the face, compositions comprising unmodified silicone resin as film forming agent are known, such as the compound having the INCI name: Trimethylsiloxysilicate or a compound having the INCI name: Polypropylsilsesquioxane, or alternatively a silicone acrylate copolymer such as the product having the INCI name: Acrylates/polytrimethylsiloxymethacrylate copolymer). Long-lasting makeup products for the eyelashes and/or the eyebrows (mascaras, eyeliners) use, for their part, typically use waxes or film-forming polymer particles in aqueous suspension of the latex type (i.e.: styrene/acrylate copolymers) as film formers.

Besides these long-lasting products, the current trend is towards semi-permanent makeup. Specifically, in recent years, conventional makeup products have met with competition from the market of semi-permanent makeup in professional salons. It is encountered in the sector of makeup for the eyes (semi-permanent mascara, permanent eyelash makeup, eyelash extensions, etc.), for the eyebrows (semi-pigmentation known as micro-blading), for the complexion (freckles, beauty spots or the whole face, glowing or healthy-complexion effect) or for the lips (semi-permanent tattooing). This new trend is driving consumers towards seeking increasingly long staying power for greater practicality (avoiding having to apply and remove makeup daily, healthy complexion effect immediately on waking up, etc.).

The aim of the present invention is to propose compositions which offer excellent staying power of the expected cosmetic effects, notably the color of the makeup on keratin materials (skin, lips, nails, hair, eyelashes, eyebrows) which may extend the duration of the cosmetic composition on keratin materials, as well as improve wear of the cosmetic composition on keratin materials and its resistance to external forces such as one or more of mechanical friction, water, sweat and perspiration, sebum, oil, etc.

In addition, the aim of the present invention is to propose compositions which afford staying power of the expected cosmetic effects, notably the color of the makeup on keratin materials, combined with a good level of comfort in comparison with conventional systems, in particular based on unmodified silicone resin.

Possibly related patent references include:
U.S. Pat. No. 11,253,462 discloses low-wax compositions containing silicone polymers among other ingredients;
US 2020/0022898 discloses film forming compositions comprising a first anionic or non-ionic non-crosslinking polyacrylate random copolymer and a second cationic non-crosslinking polyamide random copolymer;
U.S. Pat. No. 11,033,478 discloses a self-curling mascara composition containing at least about 7 percent by weight of a non-ionic water-soluble or water dispersible copolymer that includes a cyclic amide monomer, a cyclic amine monomer, and an acrylamide monomer;
U.S. Pat. No. 11,253,462 discloses compositions containing polymethyl methacrylate polymers among other ingredients;
US 2003/0082221 discloses compositions containing pullulan;
US 2016/0113860 discloses compositions containing silicone-modified pullulan and silicone oils which it thickens;
JP-A H08-208989 is discussed in the previous application, and characterized as disclosing silicone-modified pullulan which thickens silicone oil components, but which has an inadequate thickening effect;
CN 111801092 discloses compositions for impregnating a material, where the compositions contain PEG or polyethyer modified-silicones as well as dendrimer type silioxanes;
FR 3,104,958 discloses compositions containing polyurethane dispersions among other ingredients; and
FR 3,110,849 discloses compositions containing polyurethane and/or acrylic dispersions among other ingredients, with little or no fatty material.

There remains a need for improved compositions such as emulsion compositions having improved properties with respect to composition ease of and/or comfort of application of the composition, and/or in particular with respect to improved properties related to improved wear of the cosmetic composition on keratin materials and its resistance to external forces such as mechanical friction, water, sweat and perspiration, sebum and/or oil.

### SUMMARY OF THE INVENTION

The present invention relates to water-in-oil (w/o) emulsion compositions comprising at least one hydrophobically-modified pullulan compound and at least one aqueous phase film former. Preferably, the composition is a composition for application to skin. Most preferably, the composition is a foundation. Also preferably, the composition further comprises at least one thickening agent, at least one colorant, and/or at least one active agent.

The present invention also relates to methods of treating, caring for and/or making up keratinous material such as skin by applying compositions of the present invention to the keratinous material in an amount sufficient to treat, care for and/or make up the keratinous material. Preferably, the composition is a composition for application to skin. Most preferably, the composition is a foundation.

The present invention also relates to methods of making a water-in-oil (w/o) emulsion composition comprising combining at least one hydrophobically-modified pullulan compound and at least one aqueous phase film former during formation of the emulsion composition. Preferably, the composition is a composition for application to skin. Most preferably, the composition is a foundation. Also preferably, the composition further comprises at least one thickening agent, at least one colorant, and/or at least one active agent.

### DETAILED DESCRIPTION OF THE INVENTION

In the following description of the invention and the claims appended hereto, it is to be understood that the terms used have their ordinary and accustomed meanings in the art, unless otherwise specified.

“About” as used herein means within 10% of the indicated number (e.g. “about 10%” means 9%-11% and “about 2%” means 1.8%-2.2%).

“A” or “an” as used herein means “at least one.”

“At least one” means one or more and thus includes individual components as well as mixtures/combinations.

As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc.

“Film former”, “film-forming polymer” or “film-forming agent” as used herein means a polymer or resin that leaves a film on the substrate to which it is applied, for example, after a solvent accompanying the film former has evaporated, absorbed into and/or dissipated on the substrate.

“Wax” as used herein is a lipophilic fatty compound that is solid at ambient temperature (25° C.) and changes from the solid to the liquid state reversibly, having a melting temperature of more than 30° C. and, for example, more than 45° C., and a hardness of more than 0.5 MPa at ambient temperature.

“Surfactant” and “emulsifier” are used interchangeably throughout this specification.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 115° C.

“Non-volatile”, as used herein, means having a flash point of greater than about 115° C.

“Polymer” as used herein means a compound which is made up of at least two monomers.

“Matte” in compositions as used herein refers to compositions having little to no light reflection. For example, matte compositions can have average gloss properties, measured at 60°, of less than or equal to 10, for example 9, preferably less than or equal to 8, 6, 5, 4 or 1, including all ranges and subranges therebetween such as 1-10, 1-5, 2-10, 3-8, less than 1, less than 3, less than 5, etc. Such measurements can be made by depositing films to be tested onto a substrate (ex. a black scrub panel P121-10N or opacity card BYK No. 2810) using a drawdown bar (for example, of 1 mil, 3 mil, or 6 mil thickness) and an Automatic Drawdown Machine. The films can then be dried at room temperature and analyzed using a gloss meter (BYK: micro-TRI-gloss) at a desired angle, such as 60° or 85°.

“Free” or “substantially free” or “devoid of” as it is used herein means that while it is preferred that no amount of the specific component be present in the composition, it is possible to have very small amounts of it in the compositions of the invention provided that these amounts do not materially affect at least one, preferably most, of the advantageous properties of the conditioning compositions of the invention. Thus, for example, “free of phenylated silicone oil” means that an effective amount (that is, a matte-inhibiting effective amount) of phenylated silicone oil is omitted from the composition (that is, about 0% by weight), “substantially free of phenylated silicone oil” means that phenylated silicone oil is present in amounts not greater than 0.25% by weight, and “devoid of phenylated silicone oil” means that phenylated silicone oil is present in amounts not greater than 0.5% by weight, based on the total weight of the composition. The same nomenclature applies for all other ingredients identified throughout the application and in this paragraph such as, for example, (1) mattifying fillers (compositions of the invention which are “free of mattifying fillers,” “substantially free of mattifying fillers,” and “devoid of mattifying fillers,”) and (2) additional film formers other than hydrophobically-modified pullulan compound and aqueous phase film former (compositions of the invention which are “free of additional film formers,” “substantially free of additional film formers,” and “devoid of additional film formers”) have meanings consistent with the discussion within this paragraph, even if not specifically discussed for each identified ingredient. Discussed examples of the use of such language such as those in this paragraph are intended to be exemplary, not limiting.

“Makeup Result” as used herein, refers to compositions where color remains the same or substantially the same as at the time of application, as viewed by the naked eye, after an extended period of time. “Makeup Result” may be evaluated by evaluating long wear properties by any method known in the art for evaluating such properties. For example, long wear may be evaluated by a test involving the application of a composition to keratin materials such as lips and evaluating the color of the composition after an extended period of time. For example, the color of a composition may be evaluated immediately following application to keratin materials such as lips and these characteristics may then be re-evaluated and compared after a certain amount of time. Further, these characteristics may be evaluated with respect to other compositions, such as commercially available compositions.

“Keratinous material” or “keratin material” means natural nails, lips, skin such as the face, the body, the hands, and the area around the eyes, and keratin fibres such as head hair, eyelashes, eyebrows, bodily hair of a human, as well as synthetic additions such as false eyelashes, false eyebrows, false nails, etc.

“Physiologically acceptable” means compatible with keratinous material and having a pleasant color, odor and feel, and which does not cause any unacceptable discomfort (stinging or tautness) liable to discourage a consumer from using the composition. Acceptable pH levels for the internal aqueous phase of the compositions of the present invention are preferably acidic, that is, less than 7, preferably 6.5 or less, preferably 6.0 or less, preferably 5.5 or less, including all ranges and subranges therebetween such as, for example 3 to 5, 4 to 6, 5.5 to 6.5, 3 to 4.5, etc. Compositions of the present invention may be in the form of a gel composition.

“Natural compound” refers to any compound derived directly from a natural substance such as a plant without having undergone any chemical modification.

“Compound of natural origin” refers to any compound derived from a natural compound which has undergone one or more chemical modifications, for example by organic synthesis reaction, without the properties of the natural compound having been modified.

“Synthetic compound” refers to any compound which is not a natural compound or a compound of natural origin.

“Room temperature” means 25° C.

“Atmospheric pressure” means 760 mmHg, i.e. 105 pascals.

The compositions and methods of the present invention can comprise, consist of, or consist essentially of the essential elements and limitations of the invention described herein, as well as any additional or optional ingredients, components, or limitations described herein or otherwise useful. For example, the aqueous phase solvent system can “consist essentially of” water and C2-C5 monoalcohol, and/or the film forming system can consist essentially of the at least one pullulan compound and the at least one cyclic amide-containing polymer.

For purposes of the present invention, the “basic and novel property” associated with compositions, components and methods which “consist essentially of” identified ingredients or actions is “sebum resistance.”

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

All U.S. patents or patent applications disclosed herein are expressly incorporated by reference in their entirety.

### Aqueous Phase Film Former

According to the present invention, compositions including one or more aqueous phase film-forming polymers (“first film-forming polymers” or “first film formers”) that is/are non-ionic water-soluble or water dispersible polymer are provided. For clarity, by “non-ionic water-soluble or water-dispersible” it is meant that the polymer is non-ionic. The polymer is also water-soluble or water-dispersible, particularly to the extent that it can be readily stabilized throughout a vehicle (e.g., water) present in the composition.

The one or more of these first film-forming polymers are part of a aqueous phase film-forming system. The aqueous phase film forming system can comprise, consist essentially of, or consist of one aqueous phase film former.

Examples of aqueous phase film forming polymer include, but are not limited to, homopolymers and copolymers containing at least one (meth)acrylic acid ((meth)acrylate) monomer and/or at least one vinylpyrrolidone monomer such as polyvinylpyrrolidone, vinyl pyrrolidone/vinyl acetate copolymers, vinyl pyrrolidone/acrylic acid copolymers, vinyl pyrrolidone/acrylate copolymers, alkyl (e.g., butyl) acrylate/hydroxyalkyl (e.g., propyl) dimethicone acrylate copolymers, styrene/acrylates/ammonium methacrylate copolymers, acrylamide/sodium acryloyldimethyltaurate copolymer, etc.; water-soluble polysaccharides such as pectin, unmodified pullulan or cellulose compounds; polyurethanes; latexes; and mixtures thereof.

According to preferred embodiments, the at least one aqueous phase film forming polymer is at least one cyclic amide-containing polymer. Preferably, the at least one cyclic amide-containing polymer also has at least one of cyclic amine and/or acrylamide functionality as well, preferably both of these, although it is possible for the at least one polymer to contain neither cyclic amine nor acrylamide functionality.

Cyclic amide and cyclic amine monomers useful in the first film-forming polymers include those having one or more aromatic or aliphatic ring structures. These rings may have sizes ranging from about 3 to 10, for example, 5 to 8 ring members.

In certain embodiments, monomers useful in forming these first film-forming polymers are polymerizable, ethylenically-unsaturated monomers having a cyclic amine residue or a cyclic amide residue. Accordingly, the cyclic amide monomers of these first film-forming polymers may include cyclic amide residues that are or include heterocyclic ring structures such as lactams and the like. These may include α-Lactam, β-lactam, γ-lactam, δ-lactam, and ε-lactam. In one preferred embodiment, the cyclic amide is a pyrrolidone (a γ-lactam), in particular vinylpyrrolidone

Useful cyclic amine residues may include any of various heterocyclic amines such as azoles, pyrroles, pyrrolidines, carbamates, and the like. In one preferred embodiment, the cyclic amine residue is an imidazole.

In certain embodiments, acrylamide monomers useful in these first film-forming polymers include those having —C3H5NO functional groups. Examples include (meth) acrylamides.

As noted above, at least one cyclic amide-containing polymer preferably has at least one of cyclic amine and acrylamide functionality as well, preferably both, although it is possible that the polymer contains neither functionality. For example, polymer(s) containing cyclic amide residue(s) but not cyclic amine or acrylamide functionality include but are not limited to vinylpyrrolidone homopolymers (polyvinylpyrrolidone or PVP, such as that sold by BASF under the name Luviskol 30 or by Ashland under the name PVP K30L) and vinylpyrrolidone copolymers containing at least one monomer other than vinylpyrrolidone such as, for example, substituted or unsubstituted C2 (acrylic acid) or C3 (allyl) groups, possibly in the form of esters (for example, acrylates or methacrylates) or ethers, such as vinylpyrrolidone/vinyl acetate (VP/VA) copolymer such as that sold under the name PVP/VA S 630 L by Ashland.

In certain embodiments, the at least one cyclic amide-containing polymer has a weight average molecular weight in a range from about 10,000 daltons to about 1,000,000 daltons, including all ranges and subranges therebetween such as, for example, 50,000 daltons to 500,000 daltons, 75,000 daltons to 300,000 daltons, and 100,000 daltons to 250,000 daltons.

In certain embodiments, the at least one cyclic amide-containing polymer is a copolymer of N-vinyl pyrrolidone, methacrylamide, and N-vinylimidazole.

In certain embodiments, the at least one cyclic amide-containing polymer may be a commercially available variety, such as LUVISET CLEAR AT3, commercially available from BASF of Ludwigshafen, Germany.

According to preferred embodiments, the compositions of the present invention are devoid of, free of, or substantially free of, polyurethanes.

Preferably, the at least one aqueous phase film former is present in the compositions of the present invention in an amount ranging from about 0.5% to about 25% by weight, preferably from about 1% to about 20% by weight, preferably from more than 2% to about 20%, and preferably from about 2.5% to about 15% by weight with respect to the weight of the composition, including all ranges and subranges therebetween such as, for example, 4% to 15%, 2.5% to 15%, 3% to 25%, etc. Preferably, the at least one aqueous phase film former is present in the compositions of the present invention in an amount greater than 1.5% by weight of the total weight of the composition.

### Hydrophobically-Modified Pullulan Compound

According to the present invention, compositions including one or more hydrophobically-modified pullulan compound(s) (“second film-forming polymers” or “second film formers”) are provided. The one or more of these second film-forming polymers are part of a film-forming system along with the at least one cyclic amide-containing polymer. Optionally, other film forming agents can be in the film-forming system in the composition, if desired.

Pullulan is a natural polysaccharide produced from starch by the fungus Aureobasidium pullulans. Pullulan is an alpha-glucan mainly constituted of maltotriose repeating units linearly joined through alpha 1,6-glycosidic linkages. Natural pullulan is readily soluble in cold or warm water and forms clear, viscous solutions in it.

Pullulan may be produced, for example, from the fermentation of partially hydrolyzed starch or by using sucrose, sugar cane, milk, potato and other sources for carbohydrate starting materials.

Preferably, hydrophobically-modified pullulan compounds of the present invention are oil-soluble. For example, preferred pullulan compounds of the present invention have been modified with a hydrophobic group such as a siloxane group or a hydrocarbon group which imparts oil-solubility to the compound. Such modifications can include, for example, a silicone group such as a siloxysilyl group (—[—SiRR′—SiOR″R′″—]—), where R, R′, R″ and R″ are alkyl groups preferably containing 1 to 8 carbon atoms, preferably 1 to 4 carbon atoms and preferably 1 to 3 carbon atoms), or the can include C8 or greater hydrocarbon groups (for example, hydrocarbon groups containing 8 to 24 carbon atoms including ranges and subranges therebetween such as 12 to 22 carbon atoms and 12 to 18 carbon atoms).

A preferred siloxane-modified pullulan compound has a structure corresponding to:
pullulan-linker-siloxane group;
where the siloxane group is preferably a C1-C6 alkyl-substituted siloxysilyl group such as, for example, a monoalkyl-substituted siloxysilyl group, a diakyl-substituted siloxysilyl group, or a trialkyl-substituted siloxysilyl group, where the C1-C6 alkyl group is linear, cyclic or branched, substituted or unsubstituted; and
where the linker does not adversely affect either the film forming properties or the oil-solubility properties of the compound. Preferably, the linker is alkyl (—C—), ester or ether (—O—), amide or amine (—N—) or a combination thereof such as a carbamoyl group. Preferably, the linker is a carbamoyl group.

Preferably, the hydrophobically-modified pullulan compound is a trialkylsiloxysilylcarbamoyl pullulan, with a particularly preferred compound being trimethylsiloxysilylcarbamoyl pullulan.

Preferably, hydrophobically-modified pullulan compound(s) in the compositions of the present invention have a molecular weight of 50,000 to 10,000,000 daltons, preferably 100,000 to 5,000,000 daltons, preferably 250,000 to 1,000,000 daltons, including all ranges and subranges therebetween including, for example, 200,000 to 7,600,000 daltons, 350,000 to 8,750,000 daltons, 225,000 to 700,000 daltons, etc.

Preferably, the at least one hydrophobically-modified pullulan compound is present in the compositions of the present invention in an amount ranging from about 0.5% to about 25% by weight, preferably from about 1% to about 20% by weight, preferably from about 2.5% to about 17.5%, and preferably from about 5% to about 15% by weight with respect to the weight of the composition, including all ranges and subranges therebetween such as, for example, 4% to 14%, 1% to 15%, 5% to 25%, etc.

According to preferred embodiments, the at least one aqueous phase film former and the at least one hydrophobically-modified pullulan compound are present in the compositions of the present invention in a weight ratio of about 3:1 to about 1:3, preferably about 2:1 to about 1:2, and preferably about 1:1.

### Aqueous Phase

According to preferred embodiments of the present invention, compositions comprising an aqueous phase solvent system comprising (i) water in an amount of at least about 5% by weight with respect to the total weight of the composition; and (ii) optionally, at least one C2-C5 monoalcohol are provided. Preferably, the composition is in the form of an emulsion with a continuous outer phase which is oil, preferably a water-in-oil (w/o) emulsion.

According to preferred embodiments, the compositions of the present invention comprise water. Preferably, the compositions comprise at least about 10% water by weight, preferably greater than 20% water by weight, preferably greater than about 25% water by weight, and preferably greater than about 30% water by weight, preferably in amounts from about 10% to about 80%, preferably from greater than 20% to about 75%, preferably from about 25% to about 60%, and preferably from about 30% to about 50%, by weight, based on the total weight of the composition, including all ranges and subranges therebetween such as, for example, about 30% to about 60%.

Suitable C2-C5 monoalcohols, if present, include ethanol, propanol, butanol, pentanol, isopropanol, isobutanol and isopentanol. Ethanol is particularly preferred.

Preferably, the C2-C5 monoalcohol(s), if present, is/are present in the compositions of the present invention in an amount ranging from about 1% to about 50%, preferably from about 2.5% to about 40%, preferably from about 5% to about 30%, and preferably from about 5% to about 25%, by weight, based on the total weight of the composition, including all ranges and subranges in between such as, for example, about 1 to about 25%.

Preferably, the aqueous phase solvent system of the compositions of the present invention consists essentially of, or consists of, water and, optionally, C2-C5 monoalcohols. Preferably, the aqueous phase solvent system is “free of,” “devoid of” or “substantially free of” solvents other than water and, optionally, C2-C5 monoalcohols.

### Oil Phase

According to the present invention, compositions comprising at least one oil are provided. Compositions of the present invention preferably comprise sufficient oil to form a water-in-oil emulsion, preferably from about 5% to about 80% oil, more preferably from about 10% to about 60% oil, and more preferably from about 15% to about 40% oil by weight with respect to the total weight of the composition, including all ranges and subranges therebetween.

Suitable oils include volatile and/or non-volatile oils. Such oils can be any acceptable oil including but not limited to silicone oils and/or hydrocarbon oils.

According to certain embodiments, the oil carrier comprises one or more volatile silicone oils. Examples of such volatile silicone oils include linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |

Further, a volatile linear silicone oil may be employed in the present invention. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to other embodiments, the oil carrier comprises one or more non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, and for example, the oils sold under the trade names of Isopar or Permethyl. Preferably, the volatile non-silicone oils have a flash point of at least 40° C.

Non-limiting examples of volatile non-silicone volatile oils are given in Table 2 below.

TABLE 2

| Compound | Flash Point (° C.) |
|---|---|
| Isododecane | 43 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |
| Undecane | 62 |
| Tridecane | 94 |

The volatility of the solvents/oils can be determined using the evaporation speed as set forth in U.S. Pat. No. 6,338,839, the contents of which are incorporated by reference herein.

According to other embodiments of the present invention, the oil carrier comprises at least one non-volatile oil. Examples of non-volatile oils that may be used in the present invention include, but are not limited to, polar oils such as:
hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol 810, 812 and 818 by the company Dynamit Nobel;
synthetic oils or esters of formula R₅COOR₆ in which R₅ represents a linear or branched higher fatty acid residue containing from 1 to 40 carbon atoms, including from 7 to 19 carbon atoms, and R₆ represents a branched hydrocarbon-based chain containing from 1 to 40 carbon atoms, including from 3 to 20 carbon atoms, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;
synthetic ethers containing from 10 to 40 carbon atoms;
C8 to C26 fatty alcohols, for instance oleyl alcohol, cetyl alcohol, stearyl alcohol, and cetearly alcohol; and
mixtures thereof.

Further, examples of non-volatile oils that may be used in the present invention include, but are not limited to, non-polar oils such as branched and unbranched hydrocarbons and hydrocarbon waxes including polyolefins, in particular Vaseline (petrolatum), paraffin oil, squalane, squalene, hydrogenated polyisobutene, hydrogenated polydecene, polybutene, mineral oil, pentahydrosqualene, and mixtures thereof.

According to preferred embodiments, the compositions of the present invention are devoid of, free of, or substantially free of, silicone oils.

According to preferred embodiments, the compositions of the present invention are devoid of, free of, or substantially free of, waxes.

### Low HLB Surfactant

According to preferred embodiments of the present invention, compositions comprising at least one low HLB surfactant are provided. “HLB” refers to the “hydrophilic-lipophilic balance” associated with emulsifiers/surfactants. In particular, “HLB” value relates to the ratio of hydrophilic groups and lipophilic groups in emulsifiers, and also relates to solubility of the emulsifiers. Lower HLB emulsifiers are typically more soluble in oils (lipophilic material) and are more appropriate for use in water-in-oil (W/O) emulsions. Higher HLB emulsifiers are typically more soluble in water (hydrophilic material) and are more appropriate for oil-in-water (O/W) emulsions. In the context of the present invention, “low HLB surfactant” means a surfactant having an HLB value of less than or equal to 6, preferably less than 5, and preferably less than 4. Preferably, the HLB value is 3 or greater.

The low HLB value surfactant is present as all or part of a “surfactant component” in the compositions of the present invention. According to preferred embodiments of the present invention, the surfactant component has an HLB value of less than or equal to 6, preferably less than 5, and preferably less than 4. Preferably, the HLB value is 3 or greater.

According to preferred embodiments, the low HLB surfactant is nonionic.

According to preferred embodiments, the low HLB surfactant comprises a backbone and pendant group(s), wherein (1) the backbone is hydrophobic and one or more pendant groups is/are hydrophilic, or (2) the backbone is hydrophilic and one or more pendant groups is/are hydrophobic. The backbone can be silicone-based or hydrocarbon based. By “backbone,” it is meant that the surfactant comprises a main chain. By “pendant” group(s), it is meant that one or more groups is attached to the backbone or main chain of the surfactant. The pendant group(s) can be silicone-based or hydrocarbon based, and can be attached at any location along the backbone or main chain, for example at one or both terminal ends of the chain, at location(s) not at a terminal end of the chain, or both. According to preferred embodiments, the pendant group(s) include one or more ester group(s) attached to the backbone or main chain.

A preferred backbone is a hydrophobic such as, for example, one or more C8-C24 fatty compounds, preferably C12-C20 fatty compound(s), and preferably C16-C18 fatty compound(s) such as, for example, stearate, isostearate, laurate, etc, and a preferred pendant group for such a backbone is a hydrophilic group such as a polyethylene glycol (PEG) polymer or a polyglyceryl polymer.

Another preferred backbone is a hydrophilic, hydrocarbon-based backbone such as a polyethylene glycol (PEG) polymer or a polyglyceryl polymer, and a preferred pendant group for such a backbone is a hydrophobic group such as, for example, C8-C24 fatty compound(s), preferably C12-C20 fatty compound(s), and preferably C16-C18 fatty compound(s) such as, for example, stearate, isostearate, laurate, etc.

The low HLB surfactant of the present invention can be a silicone surfactant, preferably comprising dimethicone (for example, PEG-10 dimethicone or lauryl PEG-9 polydimethylsiloxyethyl dimethicone), or a non-silicone surfactant, free of dimethicone (for example, PEG-30 dihydroxystearate). The low HLB surfactant of the present invention can have alkoxylation (for example, ethoxylation or propoxylation) through groups such as, for example, (C₂H₄O)—/polyethylene glycol groups (PEG), (C₃H₆O)—/polypropylene glycol groups (PPG), etc. Suitable emulsifiers include alkoxylated compounds, partial glycerides of alkoxylated compounds, polyglycerolated compounds, and mixtures thereof, etc. Polyglycerolated compounds are preferred.

Suitable polyglycerolated compounds include, but are not limited to, compounds preferably containing 2 to 20 glyceryl groups, preferably 3 to 15 glyceryl groups, and preferably 4 to 10 glyceryl groups. Polyglyceryl-4 Diisostearate/Polyhydroxystearate/Sebacate, sold under the tradename Isolan GPS by Evonik, is particularly preferred.

Particularly preferred low HLB surfactants are as follows:

| Surfactants | HLB |
|---|---|
| POLYGLYCERYL-4 | 5 |
| DIISOSTEARATE/POLYHYDROXYSTEARATE/SEBACATE | |
| POLYGLYCERYL-4 ISOSTEARATE | 5 |
| POLYGLYCERYL-2 DIISOSTEARATE | 5.5 |
| GLYCOL DISTEARATE | 5 to 6 |
| GLYCERYL STEARATE | 5.8 |
| PEG-30 DIPOLYHYDROXYSTEARATE | 5.5 |
| PEG-10 DIMETHICONE | 4.5 |
| LAURYL PEG-9 POLYDIMETHYLSILOXYETHYL | 3 |
| DIMETHICONE | |
| PEG-9 POLYDIMETHYLSILOXYETHYL DIMETHICONE | 4 |
| PEG-6 DIISOSTEARATE | 5 |

The at least one low HLB surfactant is preferably present in the compositions of the present invention in an amount of about 1% to about 15%, preferably from about 1.5% to about 10%, and preferably from about 2% to about 8% by weight with respect to the total weight of the composition, including all ranges and subranges therebetween.

### Thickening Agent/Colorant/Active Agent

### Thickening Agent

According to preferred embodiments of the present invention, compositions further comprising at least one thickening agent are provided. Suitable thickening agents include agents for thickening an oil phase of a composition, agents for thickening a water phase of a composition, and mixtures thereof.

According to preferred embodiments, the thickening agent is a gelling agent for gelling water (water phase) in the composition. Suitable gelling agents include any gelling agent, such as for example, clays and acrylic acid (co)polymers, such as high molecular weight homo- or co-polymers comprising acrylic acid, optionally crosslinked with a polyalkenyl polyether, including some polymers identified as “carbomer” as well as amphiphilic polymers. Preferably, the gelling agent is an amphiphilic polymer or a clay, or a combination thereof.

According to preferred embodiments, the gelling agent(s) may be at least one clay. Among the clays, mention may be made of clays of the smectite family, such as laponite, of the kaolinite family, such as kaolinite, dickite, nacrite, optionally modified clays of the halloysite, dombassite, antigorite, berthierine, pyrophyllite, montmorillonite, beidellite, vermiculite, talc, stevensite, hectorite, saponite, chlorite, sepiolite and illite family.

Clays are products that are already well known per se, which are described, for example, in the publication “Mineralogie des argiles” [“Clay Mineralogy”], S. Caillère, S. Henin, M. Rautureau, 2nd Edition 1982, Masson, the teaching of which is included herein by way of reference.

Natural clay is a sedimentary rock composed to a large extent of specific minerals, silicates generally of aluminium. Kaolin is thus a natural clay.

The clays may also be synthetic. Thus, Sumecton mentioned below is a synthetic saponite.

The clays may also be chemically modified with various compounds, such as acrylic acids, polysaccharides (for example carboxymethylcellulose) or organic cations.

Preferably, in the context of the present invention, use is made of clays that are cosmetically compatible and acceptable with keratin materials. Clays that may especially be mentioned include kaolinite, montmorillonites, saponites, laponites, hectorites, and illites. Mixtures of clays and natural clays may also be used.

Natural clays that may be mentioned include green clays, in particular rich in illite; clays rich in montmorillonite, known as fuller's earth, or such as bentonite or else white clays rich in kaolinite. As bentonites, or modified bentonites, mention may in particular be made of those sold under the names Bentone 38 VCG®, Bentone Gel CAO VR, Bentone 27 VR, Bentone Gel ISD V, and Bentone Gel MIO V® by the company Elementis.

By way of clay rich in montmorillonite, mention may be made of the aluminium silicate hydrate sold under the name Gel White H® by the company Rockwood.

By way of saponite, which belongs to the montmorillonite family, mention may be made of synthetic saponite, in particular the product sold by the company Kunimine under the name Sumecton®.

By way of tack, mention may be made of those sold under the names Rose Talc® and Talc SG-2000® sold by the company Nippon Talc, Luzenac Pharma M® sold by the company Luzenac, J-68BC® from US Corporation and Micro ACE-P-3® sold by the company Nippon Talc.

Preferably, if present, the thickening agent(s) is/are present in the compositions of the present invention in amounts ranging from about 0.1 to about 30% by weight, preferably from 0.5 to 25% by weight, preferably from 1 to 20% and preferably from 2.5 to 15% by weight, all weights based on the weight of the composition as a whole, including all ranges and subranges therebetween such as, for example, 0.1 to 1.5%, 2 to 20%, 10 to 20%, etc.

### Active Agent

According to preferred embodiments of the present invention, compositions further comprising at least one active agent are provided. Preferably, the active agent is in the aqueous phase (hydrophilic active agent), although active agents may be present in the oil phase (hydrophobic active agent).

In some embodiments, the active agent may be:
a moisturizing agent, such as a polyol such as, for example, glycerin and sugars, urea and its derivatives, such as in particular hydroxyalkyl urea, in particular hydroxyalkylurea, and mixtures thereof;
a desquamating agent, which may be a compound capable of acting either directly on desquamation by promoting exfoliation, such as β-hydroxy acids, in particular salicylic acid and its derivatives (including 5-n-octanoylsalicylic acid); α-hydroxy acids, such as glycolic acid, citric acid, lactic acid, tartaric acid, malic acid or mandelic acid; urea; gentisic acid; oligofucoses; cinnamic acid; extract of Saphora japonica; resveratrol and certain jasmonic acid derivatives; or acting on the enzymes involved in the desquamation or degradation of corneodesmosomes, glycosidases, stratum corneum chymotryptic enzyme (SCCE), or even other proteases (trypsin, chymotrypsin-like). Mention may be made of agents for chelating mineral salts: EDTA; N-acyl-N,N′,N′-ethylenediaminetriacetic acid; aminosulfonic compounds and in particular (N-2-hydroxyethylpiperazine-N-2-ethane) sulfonic acid (HEPES): 2-oxothiazolidine-4-carboxylic acid (procysteine) derivatives; α-amino acid derivatives of the type such as glycine (as described in EP-0 852 949 and sodium methylglycinediacetate sold by BASF under the trade name Trilon M); honey; sugar derivatives such as O-octanoyl-6-D-maltose and N-acetylglucosamine;
a humectant;
an anti-aging agent, which may include, for example, one or more of C-beta-D-xylopyranoside-2-hydroxypropane (Pro-Xylane), retinol, peptides, caffeine, and other components that provide improvement to skin texture, any other suitable soluble/dispersible targeted active ingredient, and combinations thereof.
a mattifying agent, which may include, but is not limited to, mattifying fillers such as, for example, talc, silica, silicone elastomers, and polyamides, and waxes such as, for example, beeswax and copernicia cerifera (carnauba) wax.
an antimicrobial agent, non-limiting examples of which include 2,4,4′-trichloro-2′-hydroxydiphenyl ether (or triclosan). 3,4,4′-trichlorobanilide, phenoxyethanol, phenoxypropanol, phenoxyisopropanol, hexamidine isethionate, metronidazole and its salts, micronazole and its salts, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and its salts, benzoyl peroxide, 3-hydroxybenzoic acid, 4-hydroxybenzoic acid, phytic acid, N-acetyl-L-cysteine acid, lipoic acid, azelaic acid and its salts, arachidonic acid, resorcinol, 2,4,4′-trichloro-2′-hydroxydiphenyl ether, 3,4,4′-trichlorocarbanalide, octopirox, octoxyglycerine, octanoylglycine, caprylyl glycol, 10-hydroxy-2-decanoic acid, dichlorophenylimidazole dioxolane and its derivatives described in patent WO 93/18743, farnesol and phytosphingosines, and mixtures thereof;
a pigment modifying agent and/or skin lightening agents, such as double-stranded RNA oligonucleotides are useful for decreasing tyrosinase expression. Mention may also be made of ceramides, vitamin C and derivatives thereof, in particular vitamin CG, CP and 3-O ethyl vitamin C, alpha- and beta-arbutin, ferulic acid, kojic acid, resorcinol and derivatives thereof, calcium D-pantetheine sulphonate, lipoic acid, ellagic acid, vitamin B3, phenylethyl resorcinol, for instance Symwhite 377® from the company Symrise, a kiwi fruit (Actinidia chinensis) juice sold by Gattefosse, an extract of Paeonia suffructicosa root, such as the product sold by the company Ichimaru Pharcos under the name Botanpi Liquid B®, an extract of brown sugar (Saccharum officinarum), such as the extract of molasses sold by the company Taiyo Kagaku under the name Molasses Liquid, a mixture of undecylenic acid and undecylenoyl phenyl alanine, such as Sepiwhite MSH® from Seppic;
an amino acid such as arginine, histidine, methionine, lysine, proline, leucine, lycine, sarcosine, etc.;
a vitamin such as a vitamin A compound such as retinol or retinoic acid, vitamin B compound such as vitamin B1, B3 or B6, in particular niacinamide, a vitamin C compound such as an ascrobyl phosphate salt (e.g., magnesium or sodium, and/or derivatives thereof;
an ultraviolet (UV) filter such as, e.g., an aminobenzoic acid derivative, a dibenzoylmethane derivative, a salicylic acid derivative, a cinnamic derivative, a β,β diphenylacrylate derivative, a benzophenone derivative, benzylidene camphor derivative, and mixtures thereof. Mention may be made especially of ethylhexyl methoxycinnamate sold under the tradename UVINUL MC 800 by the company BASF, of ethylhexyl salicylate sold under the tradename NEO HELIOPAN OS® by the company SYMRISE and of octocrylene sold under the tradename NEO HELIOPAN 3030 by the company SYMRISE; and/or
a combination thereof.

### Additional Additives

The composition of the invention can also comprise any additive usually used in the field under consideration. For example, additional film forming agents (in addition to carbosiloxane dendrimer compound and cyclic amide-containing polymer), waxes, dispersants such as poly(12-hydroxystearic acid), preserving agents, fragrances, fillers, antioxidants, neutralizing agents, silicone elastomers, and mixtures thereof can be added. A non-exhaustive listing of such ingredients can be found in U.S. patent application publication no. 2004/0170586, the entire contents of which is hereby incorporated by reference. Further examples of suitable additional components can be found in the other references which have been incorporated by reference in this application. Still further examples of such additional ingredients may be found in the International Cosmetic Ingredient Dictionary and Handbook (9^{th} ed. 2002). However, it is to be understood that preferred embodiments of the present invention include compositions which are “free,” substantially free” or “devoid” of the ingredients discussed in this paragraph such as additional film forming agents and waxes as previously-discussed as well as fillers, in particular mattifying fillers (compositions are “free of mattifying fillers,” “substantially free of mattifying fillers,” or “devoid of mattifying fillers.”

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

Needless to say, the composition of the invention should be cosmetically or dermatologically acceptable, i.e., it should contain a non-toxic physiologically acceptable medium and should be able to be applied to the skin of human beings.

According to other preferred embodiments, methods of treating, caring for and/or enhancing the appearance of skin by applying compositions of the present invention to the skin in an amount sufficient to treat, care for and/or enhance the appearance of the skin are provided. In accordance with these preceding preferred embodiments, the compositions of the present invention are applied topically to the desired area of the skin in an amount sufficient to treat, care for and/or enhance the appearance of the skin. The compositions may be applied to the desired area as needed, preferably once or twice daily, more preferably once daily and then preferably allowed to dry before subjecting to contact such as with clothing or other objects (for example, a topcoat). Preferably, the composition is allowed to dry for about 1 minute or less, more preferably for about 45 seconds or less. The composition is preferably applied to the desired area that is dry or has been dried prior to application, or to which a basecoat has been previously applied.

According to a preferred embodiment of the present invention, compositions having improved cosmetic properties such as, for example, increased water-resistance, sebum-resistance and/or transfer-resistance are provided. Preferably, the composition is a composition for skin. Most preferably, the composition is a foundation further comprising at least one colorant, preferably a surface-treated pigment.

According to a preferred embodiment of the present invention, methods of making a water-in-oil (w/o) emulsion composition comprising combining (adding) at least one hydrophobically-modified pullulan compound and at least one aqueous phase film former during preparation of the emulsion composition are provided. Preferably, the composition is a composition for skin. Preferably, the at least one hydrophobically-modified pullulan compound and at least one aqueous phase film former are combined or added in the amounts and/or ratios discussed above. Most preferably, the composition is a foundation further comprising at least one colorant, preferably a surface-treated pigment.

According to preferred embodiments of the present invention, kits comprising, as separate compositions in one or more containers within the kits, (A) at least one composition of the present invention as discussed above, in particular a foundation, comprising at least one hydrophobically-modified pullulan compound and at least one aqueous phase film former and (B) at least one other composition such as a primer coat composition, a basecoat composition, or a topcoat composition are provided. Compositions (A) and (B) may be contained in different portions or sections of the same container within the kit. However, compositions (A) and (B) may also be in different containers with the kit.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example 1—Sample W/O Emulsion Foundation Formulation

| | Total %100.00 - | Total 100% - |
|---|---|---|
| | General | Preferred |
| Chemical Name/Function | % wt/wt | % wt/wt |
| Surfactants | 1-10% | 1-5% |
| Aqueous Phase Film Former | 0.5-10% | 1-5% |
| Hydrophobically-Modified Pullulan | 0.5-10% | 1-5% |
| Oils | 5-50% | 20-40% |
| Colorants | 1-30% | 5-20% |
| Water | QS | QS |
| Monoalcohol | 1-20% | 3-10% |
| Polyol | 1-10% | 2-8% |
| Active Agent (vitamin) | 1-20% | 5-20% |
| Clays | 0.1-10% | 0.1-5% |
| Fillers | 0.5-10% | 1-5% |

### Example 2—Exemplified W/O Emulsion Foundations

| | Composition | Composition | Composition | Composition | Composition | Composition | Composition | Composition |
|---|---|---|---|---|---|---|---|---|
| INCI US | A | B | C | D | E | F | G | H |
| WATER | 36.30 | 31.95 | 27.60 | 23.25 | 35.43 | 33.69 | 35.43 | 33.69 |
| MAGNESIUM SULFATE | 0.87 | 0.87 | 0.87 | 0.87 | 0.87 | 0.87 | 0.87 | 0.87 |
| BUTYLENE GLYCOL | 2.61 | 2.61 | 2.61 | 2.61 | 2.61 | 2.61 | 2.61 | 2.61 |
| PHENOXYETHANOL | 0.17 | 0.17 | 0.17 | 0.17 | 0.17 | 0.17 | 0.17 | 0.17 |
| METHACRYLAMIDE/VINYL | — | 4.35 | 8.70 | 13.05 | — | — | — | — |
| IMIDAZOLE COPOLYMER | | | | | | | | |
| (LUVISET CLEAR AT3) | | | | | | | | |
| [symbol] VA COPOLYMER | — | — | — | — | 0.87 | 2.61 | — | — |
| (PVP/VA 630L) | | | | | | | | |
| PVP (LUVISKOL K30) | — | — | — | — | — | — | 0.87 | 2.61 |
| ALCOHOL DENAT. | 4.35 | 4.35 | 4.35 | 4.35 | 4.35 | 4.35 | 4.35 | 4.35 |
| POLYGLYCERYL-4 | 0.87 | 0.87 | 0.87 | 0.87 | 0.87 | 0.87 | 0.87 | 0.87 |
| ISOSTEARATE | | | | | | | | |
| POLYGLYCERYL-4 | 2.61 | 2.61 | 2.61 | 2.61 | 2.61 | 2.61 | 2.61 | 2.61 |
| DIISOSTEARATE/ | | | | | | | | |
| POLYHYDROXYSTEARATE/ | | | | | | | | |
| SEBACATE | | | | | | | | |
| ACETYL TRIBUTYL | 0.29 | 0.29 | 0.29 | 0.29 | 0.29 | 0.29 | 0.29 | 0.29 |
| CITRATE | | | | | | | | |
| DIMETHICONE | 1.52 | 1.52 | 1.52 | 1.52 | 1.52 | 1.52 | 1.52 | 1.52 |
| ISODODECANE | 10.73 | 10.73 | 10.73 | 10.73 | 10.73 | 10.73 | 10.73 | 10.73 |
| M HECTORITE (and) | 11.31 | 11.31 | 11.31 | 11.31 | 11.31 | 11.31 | 11.31 | 11.31 |
| PROPYLENE | | | | | | | | |
| CARBONATE | | | | | | | | |
| ISOPROPYL LAUROYL | 0.87 | 0.87 | 0.87 | 0.87 | 0.87 | 0.87 | 0.87 | 0.87 |
| SARCOSINATE | | | | | | | | |
| ISOHEXADECANE | 5.80 | 5.80 | 5.80 | 5.80 | 5.80 | 5.80 | 5.80 | 5.80 |
| [symbol] YSILYLCARBAMOYL | 8.70 | 8.70 | 8.70 | 8.70 | 8.70 | 8.70 | 8.70 | 8.70 |
| PULLULAN (TSPL-ID-30) | | | | | | | | |
| HYDROPHOBICALLY | 13.00 | 13.00 | 13.00 | 13.00 | 13.00 | 13.00 | 13.00 | 13.00 |
| MODIFIED PIGMENTS | | | | | | | | |

[symbol]

indicates data missing or illegible when filed

### Example 3—Comparison of 600 Gloss Values for Oil-Phase and Combined Oil-Phase+Water-Phase Film Former Foundations

Simplex foundation formulations were created with constant concentration of pullulan compound (TSPL) in isododecane in combination with 0.87% or 2.61% concentrations of active water-phase film-former (Luviset, VP/VA, PVP). A 6 mil drawdown bar was used to create a film of each composition A, B, D, E, F, G and H on an opacity card. The films were then dried at 32° C. in an oven and measured with a BYK Trigloss meter over the black side of the card. It was observed that formulations containing both oil-phase (TSPL) and water-phase film formers show reduced gloss values compared to oil-phase film former alone (composition A) when measured at 60° as reflected in the Table below.

| Summary Description | Composition | Gloss 60° |
|---|---|---|
| TSPL only | Composition A | 55.1 |
| TSPL + Luviset (0.87%) | Composition B | 26.4 |
| TSPL + Luviset (2.61%) | Composition D | 1 |
| TSPL + VP/VA (0.87%) | Composition E | 28.5 |
| TSPL + VP/VA (2.61%) | Composition F | 36.3 |
| TSPL + PVP (0.87%) | Composition G | 14.7 |
| TSPL + PVP (2.61%) | Composition H | 13.1 |

### Example 4—Comparison of 60° Gloss Values for Foundations with Artificial Sebum

The same simplex foundation formulations tested in example 3 were mixed 95% of the formulation+5% Artificial sebum in a speedmixer, and then a 6 mil drawdown bar was used to create a film of each on an opacity card. The films were then dried at 32° C. in an oven and measured with a BYK Trigloss meter over the black side of the card. It was observed that formulations containing both oil-phase (TSPL) and water-phase film formers show reduced gloss values compared to formulation containing oil-phase film former alone when measured at 60° as reflected in the Table below.

| | | Gloss 60° + |
|---|---|---|
| Summary Description | Composition | 5% Sebum |
| TSPL only | Composition A | 69.8 |
| TSPL + Luviset (0.87%) | Composition B | 27.8 |
| TSPL + Luviset (2.61%) | Composition D | 0.6 |
| TSPL + VP/VA (0.87%) | Composition E | 25.9 |
| TSPL + VP/VA(2.61%) | Composition F | 31.3 |
| TSPL + PVP (0.87%) | Composition G | 31.9 |
| TSPL + PVP (2.61%) | Composition H | 26 |

### Example 5—60° Gloss Values of Foundations with Increasing Concentration of Water-Phase Film-Former

Simplex foundation formulations A, B, C and D were created with constant concentration of pullulan compound (TSPL) in isododecane in combination with 0%, 0.87%, 1.74%, or 2.61% active polymer content of Luviset AT Clear 3 film-former. A 6 mil drawdown bar was used to create a film of each on an opacity card. Separate films were created from the base foundation, and a 95%/5% mixture of foundation and artificial sebum was produced. The films were then dried at 32° C. in an oven and measured with a BYK Trigloss meter over the black side of the card. It was observed that formulations containing both oil-phase (TSPL) and water-phase (Luviset) film formers show reduced gloss values compared to oil-phase film former alone (when measured at 60°). Additionally, it was observed that increasing the concentration of the water-phase film-former decreases the gloss value in a concentration-dependent manner.

| | | Gloss | Gloss 60° |
|---|---|---|---|
| Note | Composition | 60° | with 5% Sebum |
| TSPL only | Composition A | 55.1 | 69.8 |
| TSPL + Luviset (0.87%) | Composition B | 26.4 | 27.8 |
| TSPL + Luviset (1.74%) | Composition C | 2.9 | 1.2 |
| TSPL + Luviset (2.61%) | Composition D | 1 | 0.6 |

### Example 6—85° Gloss Values of Foundations with Increasing Concentration of Water-Phase Film-Former

Example 5 was repeated, with measurement at 85°. It was observed that formulations containing both oil-phase (TSPL) and water-phase (Luviset) film formers show reduced gloss values compared to oil-phase film former alone. Additionally, it was observed that increasing the concentration of the water-phase film-former decreases the gloss value in a concentration-dependent manner.

| | | Gloss | Gloss 85° + |
|---|---|---|---|
| Comment | Composition | 85° | 5% Sebum |
| TSPL only | Composition A | 65.1 | 68 |
| TSPL + Luviset (0.87%) | Composition B | 60.2 | 59.5 |
| TSPL + Luviset (1.74%) | Composition C | 29 | 4 |
| TSPL + Luviset (2.61%) | Composition D | 6.2 | 1 |

### Example 7—Dry Transfer Testing

The following compositions were prepared:

| | Compo- | Compo- |
|---|---|---|
| INCI US | sition A | sition J |
| WATER | 36.29640000 | 21.79350000 |
| MAGNESIUM SULFATE | 0.87000000 | 0.87000000 |
| BUTYLENE GLYCOL | 2.61000000 | 2.61000000 |
| PHENOXYETHANOL | 0.17400000 | 0.17400000 |
| SKINCARE INGREDIENTS | — | 5.61150000 |
| VP/METHACRYLAMIDE/VINYL | — | 8.70000000 |
| IMIDAZOLE COPOLYMER | | |
| (as Luviset Clear AT3, 20% active) | | |
| ALCOHOL DENAT. | 4.35000000 | 4.35000000 |
| SODIUM HYDROXIDE | — | 0.19140000 |
| POLYGLYCERYL-4 ISOSTEARATE | 0.87000000 | 0.87000000 |
| POLYGLYCERYL-4 | 2.61000000 | 2.61000000 |
| DIISOSTEARATE/ | | |
| POLYHYDROXYSTEARATE/SEBACATE | | |
| ACETYL TRIBUTYL CITRATE | 0.28710000 | 0.28710000 |
| DIMETHICONE | 1.52250000 | 1.52250000 |
| ISODODECANE | 10.72710000 | 7.26450000 |
| FRAGRANCE | | 0.08700000 |
| DISTEARDIMONIUM HECTORITE (and) | 11.31000000 | 11.31000000 |
| PROPYLENE CARBONATE | | |
| TRIHYDROXYSTEARIN | — | 0.26100000 |
| ISOPROPYL LAUROYL | 0.87000000 | 0.87000000 |
| SARCOSINATE | | |
| ISOHEXADECANE | 5.80290000 | 5.80290000 |
| FILLER PARTICLES | — | 3.11460000 |
| TRIMETHYLSILOXYSILYLCAR- | 8.70000000 | 8.70000000 |
| BAMOYL PULLULAN | | |
| HYDROPHOBICALLY MODIFIED | 13 | 13 |
| PIGMENTS | | |

20 uL of Foundation compositions A and J were applied in a 2.5 cm×2.5 cm square to a bare forearm and dried for 45 minutes. Foundations were then wiped 5 times in alternating directions with an ISO 105 F09 Crock square each with consistent pressure. The foundation containing both TSPL and Luviset (composition J) was found to be more resistant to disruption and transfer under rubbing compared to the foundation which did not contain Luviset (composition A).

### Example 8—Resistance to Rubbing in Presence of Sebum

The following compositions were prepared:

| | Compo- | Compo- |
|---|---|---|
| INCI Name | sition K | sition L |
| WATER | 31.89 | 12.93 |
| MAGNESIUM SULFATE | 0.70 | 0.70 |
| CAPRYLYL GLYCOL | 0.18 | 0.09 |
| PHENOXYETHANOL | 0.09 | 0.09 |
| SKINCARE INGREDIENTS | 9.68 | 9.68 |
| CELLULOSE GUM | 0.26 | 0.26 |
| VP/METHACRYLAMIDE/VINYL | — | 14.65 |
| IMIDAZOLE COPOLYMER | | |
| (Luviset Clear AT3) | | |
| FRAGRANCE | — | 0.04 |
| ALCOHOL DENAT. | 4.40 | 4.36 |
| POLYGLYCERYL-4 | 2.64 | 2.64 |
| DIISOSTEARATE/ | | |
| POLYHYDROXYSTEARATE/SEBACATE | | |
| ACETYL TRIBUTYL CITRATE | 0.29 | 0.29 |
| ISODODECANE | 4.40 | 5.72 |
| DISTEARDIMONIUM HECTORITE (and) | 13.20 | 13.20 |
| PROPYLENE CARBONATE | | |
| ISOHEXADECANE | 6.16 | 9.24 |
| FILLER PARTICLES | 2.38 | 2.38 |
| TRIMETHYLSILOXYSILYLCAR- | 11.73 | 11.73 |
| BAMOYL PULLULAN (TSPL-ID-30) | | |
| HYDROPHOBICALLY MODIFIED | 12.00 | 12.00 |
| PIGMENTS | | |

20 uL of Compositions K and L were applied to a white artificial protein leather substrate in a 3.2 cm by 3.2 cm square with a nitrile gloved finger. Samples were dried at 32° C. in an oven. Samples were then read on a spectrophotometer to obtain CIE LAB values for the initial substrate and substrate+foundation. An ISO 105 F09 Crock square was soaked in artificial sebum and any excess sebum was blotted off with a lint-free paper wipe. Samples were then rubbed twice in alternating directions with consistent force. Samples were then allowed to dry at least 6 hours and read again to obtain LAB values for the post rubbing foundation color. L values were used to calculate the normalized ΔL from the equation:

$$Normalized Δ L = \frac{ABS ( {L}_{final} - {L}_{initial} )}{ABS ( {L}_{Substrate} - {L}_{initial} )} \times 100$$

where:
ABS=absolute value
Lfinal=final L value in LAB color space of deposit
Linitial=initial L value of deposit
Lsubstrate=L value of the substrate (supplare, artificial protein leather)
Normalized ΔL=% darkness change of make-up product

It was observed that the foundation containing water-phase film-former had significantly less change in L value compared to the foundation containing TSPL only, indicating resistance to rubbing in the present of sebum, as reflected in the Table below:

| | | Average | Std. Dev. |
|---|---|---|---|
| NOTE | Composition | Normalized ΔL | Normalized ΔL |
| TSPL only | Composition J | 12.91 | 1.51 |
| TSPL + Luviset | Composition K | 0.80 | 0.69 |

What is claimed is:

1. A water-in-oil emulsion composition comprising (a) water, (b) at least one oil, (c) at least one hydrophobically-modified pullulan compound; and (d) at least one aqueous phase film former.

2. The composition of claim 1, further comprising (e) at least one compound selected from the group consisting of at least one thickening agent, at least one colorant, and at least one active agent.

3. The composition of claim 2, wherein at least two of thickening agent, colorant and active agent are present in the composition.

4. The composition of claim 2, wherein all three of thickening agent, colorant and active agent are present in the composition.

5. The composition of claim 2, wherein at least one thickening agent is present in the composition, and the at least one thickening agent is a clay, preferably a hectorite.

6. The composition of claim 2, wherein at least one coloring agent is present in the composition, and the at least one coloring agent is a surface-treated pigment.

7. The composition of claim 2, wherein at least one active agent is present in the composition, and the at least one active agent is a vitamin, preferably a vitamin B compound.

8. The composition of claim 1, wherein the at least one hydrophobically-modified pullulan compound is a siloxane-modified pullulan compound, preferably trimethylsiloxysilylcarbamoyl pullulan.

9. The composition of claim 1, wherein the at least one aqueous phase film former is at least one cyclic amide-containing polymer, preferably a copolymer of N-vinyl pyrrolidone, methacrylamide, and N-vinylimidazole, a polyvinylpyrrolidone, a vinylpyrollidone/vinyl acetate copolymer, or mixtures thereof.

10. The composition of claim 8, wherein the at least one aqueous phase film former is at least one cyclic amide-containing polymer, preferably a copolymer of N-vinyl pyrrolidone, methacrylamide, and N-vinylimidazole, a polyvinylpyrrolidone, a vinylpyrollidone/vinyl acetate copolymer, or mixtures thereof.

11. The composition of claim 1, wherein the weight ratio of aqueous phase film former to hydrophobically-modified pullulan compound is from about 3:1 to about 1:3, preferably about 1:1.

12. The composition of claim 1, wherein water is present in an amount greater than 20% by weight with respect to the total weight of the composition, preferably between about 25% and 60% by weight with respect to the total weight of the composition.

13. A method of making up skin comprising applying the composition of claim 1 to skin in an amount sufficient to make up the skin.

14. A water-in-oil emulsion composition comprising (a) water, (b) at least one volatile hydrocarbon oil, (c) at least one siloxane-modified pullulan compound; (d) at least one cyclic amide-containing polymer; (e) at least one thickening agent which is a clay; (f) at least one surface-treated pigment; and (g) at least one vitamin compound.
